(19) United States
(12) Patent Application Publication
(10) Pub. No.: US 2008/0039438 A1
(43) Pub. Date: Feb. 14, 2008

(54) 1B-Methylcarbapenem Derivative and Process for the Preparation Thereof

(75) Inventors: Kyung Ho Yoo, Seoul (KR); Dong Jin Kim, Seoul (KR); Yong Koo Kang, Seoul (KR); Kyung Seok Lee, Suwon-si (KR); Gwan Sun Lee, Seoul (KR); Maeng Sup Kim, Seoul (KR); Jae Hoon Kang, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Korea Institute of Science and Technology, Seoul (KR)

(21) Appl. No.: 11/570,468

(22) PCT Filed: Jun. 14, 2005
(86) PCT No.: PCT/KR05/01798

§ 371 Date: Dec. 12, 2006

(30) Foreign Application Priority Data
Jun. 14, 2004 (KR) 10-2004-043752

(51) Int. Cl. A61K 31/397; A61P 31/04; C07D 205/12; C07D 261/18

(52) U.S. Cl. 514/210.16; 540/200; 548/248

(57) ABSTRACT

The present invention relates to a novel 1β-methylcarbapenem derivative, a process for the preparation thereof and a pharmaceutical composition comprising the 1β-methylcarbapenem derivative or a pharmaceutically acceptable salt thereof as an active antibacterial ingredient.

### FIELD OF THE INVENTION

The present invention relates to a novel 1β-methylcarbapenem derivative, a process for the preparation thereof and a pharmaceutical composition comprising same.

### BACKGROUND OF THE INVENTION

Carbapenem antibiotics are considered to the ideal antibiotics for their broader and stronger antibacterial activities against Gram-positive and Gram-negative bacteria than cephalosporin or penicillin antibiotics, specially against the resistant bacterial strains.

Imipenem (N-formimidoly thienamycin, MK-0787), developed by Merck at 1979, is the first carbapenem antibiotic having an excellent antibacterial activity (J. Med. Chem. 1979, 22, 1435). However, it is easily degraded by the hydrolytic action of human renal dehydropeptidase-I (DHP-I) secreted in the kidney, and it must be used together with cilastatin, a DHP-I repressor. Meropenem (SM-7338), developed by Sumitomo, Japan, is a 1β-methylcarbapenem antibiotic which overcomes most of the disadvantages of imipenem (J. Antibiot. 1990, 43, 519). Meropenem shows a comparable antibacterial activity against MRSA (methicillin-resistant Staphylococcus aureus) and a more potent activity against Pseudomonas aeruginosa than imipenem, but has a shorter in vivo half-life and less potent antibacterial activity against Gram-positive bacteria than imipenem.

Also, ertapenem, commercialized by Zeneca, UK and Merck at 2001, has a long in vivo half-life and is stable toward the degradative action of ESBL (extended spectrum beta lactamase) and AmpC, but has not good antibacterial activity against Pseudomonas aeruginosa (Int. J Antimicrob. Agents 2002, 20, 136).

Accordingly, the present inventors have endeavored to develop a novel carbapenem antibiotic which is free of the drawbacks of existing antibiotics and has an excellent antibacterial activity.

### SUMMARY OF THE INVENTION

Accordingly, it is an object of the present invention to provide a novel 1β-methylcarbapenem derivative having an excellent antibacterial activity and superior stability to DHP-I.

It is another object of the present invention to provide a process for the preparation of such a 1β-methylcarbapenem derivative.

It is further object of the present invention to provide an intermediate useful for the preparation of the 1β-methylcarbapenem.

It is further object of the present invention to provide a pharmaceutical composition comprising the 1β-methylcarbapenem derivative or a pharmaceutically acceptable salt thereof as an active ingredient.

In accordance with one aspect of the present invention, there is provided the 1β-methylcarbapenem derivative of formula (I) or a pharmaceutically acceptable salt thereof.

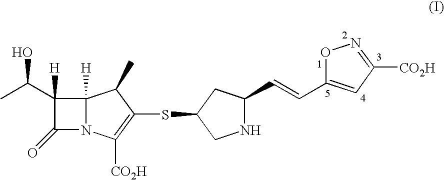

In accordance with further aspect of the present invention, there is provided a process for the preparation of the 1β-methylcarbapenem derivative or a pharmaceutically acceptable salt thereof.

In accordance with further aspect of the present invention, there is provided the thiol derivative used as an intermediate and a process for the preparation thereof.

In accordance with further aspect of the present invention, there is provided a pharmaceutical composition comprising the 1β-methylcarbapenem derivative of formula (I) or a pharmaceutically acceptable salt thereof as an active antibacterial ingredient.

### DETAILED DESCRIPTION OF THE INVENTION

The 1β-methylcarbapenem derivative of the present invention is a compound having an isoxazole with a carboxylate substituent, which is connected via a vinyl group to position 5 of the pyrrolidine moiety of 1β-methylcarbapenem.

The 1β-methylcarbapenem derivative of the present invention can also be used in the form of a pharmaceutically acceptable salt, hydrate or solvate. The pharmaceutically acceptable salt may be an alkali metal salt of the compound of formula (I), preferably a sodium salt, or an acid additional salt. The acid may be an inorganic or organic acid, e.g., hydrochloric acid, hydrobromic acid, sulfuric acid, phosphoric acid, citric acid, acetic acid, lactic acid, tartaric acid, maleic acid, fumaric acid, gluconic acid, methanesulfonic acid, glycolic acid, succinic acid, 4-toluenesulfonic acid, gluturonic acid, embonic acid, glutamic acid or aspartic acid.

As shown in Reaction Scheme (I), the inventive compound of formula (I) may be prepared from the carbapenem enolphosphate compound of formula (II) and the intermediate compound having the thiol structure of formula (III): 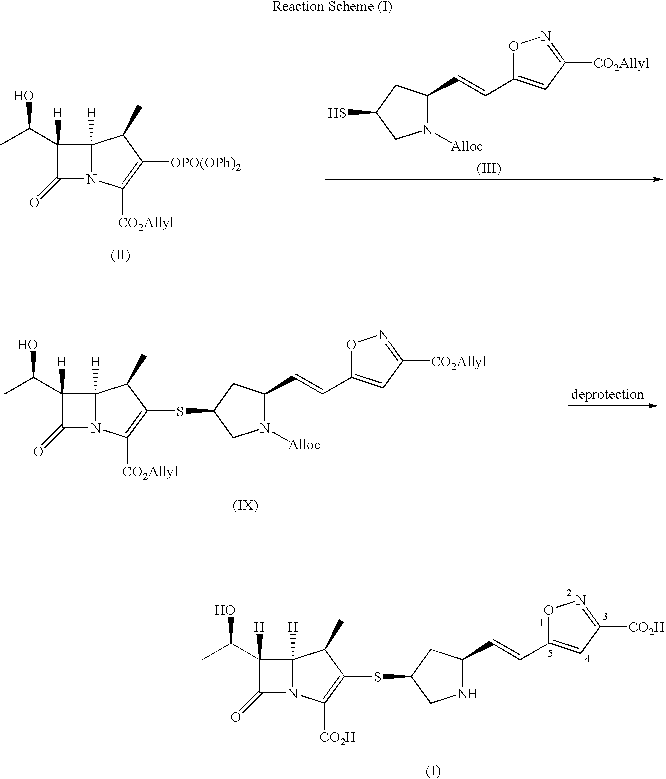
wherein,

Allyl is —CH₂—CH═CH₂ and Alloc is

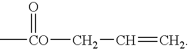

The above process comprises the steps of:

(a) reacting the compounds of formula (II) and formula (III) in the presence of a base to obtain the protected carbapenem compound of formula (IX); and

(b) subjecting the compound of formula (IX) to a deprotection reaction.

The carbapenem intermediate of formula (II) used as a starting material in step (a) can be prepared by the conventional method (Catchpole, C. R. et al. Antimicro. Agents Chemother. 1992, 36, 1928).

Specifically, the base used in step (a) may be a tertiary amine such as trimethylamine, triethylamine, N,N-diisopropylamine (DIPEA), 2,6-lutidine, picoline, N,N-dimethylaniline, pyridine and 4-dimethylaminopyridine, and N,N-diisopropylamine is preferable. This reaction can be carried out at a temperature ranging from −10 to 10° C., preferably, at 0° C. for 1 to 3 hours, preferably, 1.5 hours. The solvent used in this step is preferably acetonitrile.

In step (b), the deprotection of the protected carbapenem compound of formula (IX) can be carried out by any of the conventional methods. For example, the protecting group can be eliminated using a palladium catalyst such as tetrakis(triphenylphosphine)palladium and di(triphenylphosphine)dichloropalladium together with tributyltin hydride (n-Bu₃SnH), preferably, a combination of tetrakis(triphenylphosphine)palladium catalyst and tributyltin hydride at a temperature ranging from −10 to 10° C., preferably, at 0° C. for 1 to 3 hours, preferably, 1.5 hours. The solvent used in this reaction may be dichloromethane, a mixture of dichloromethane and water, or tetrahydrofuran, and dichloromethane is preferable.

The deprotected carbapenem compound of formula (I) may be further reacted with an alkali metal compound, preferably sodium 2-ethylhexanoate (SHE) or sodium bicarbonate, under the same deprotection condition for 10 to 60 minutes to obtain an alkali metal salt, preferably a sodium salt, of the 1β-methylcarbapenem derivative of formula (I).

The intermediate compound of formula (III) used in Reaction Scheme (I) may be prepared in accordance with Reaction Scheme (II): 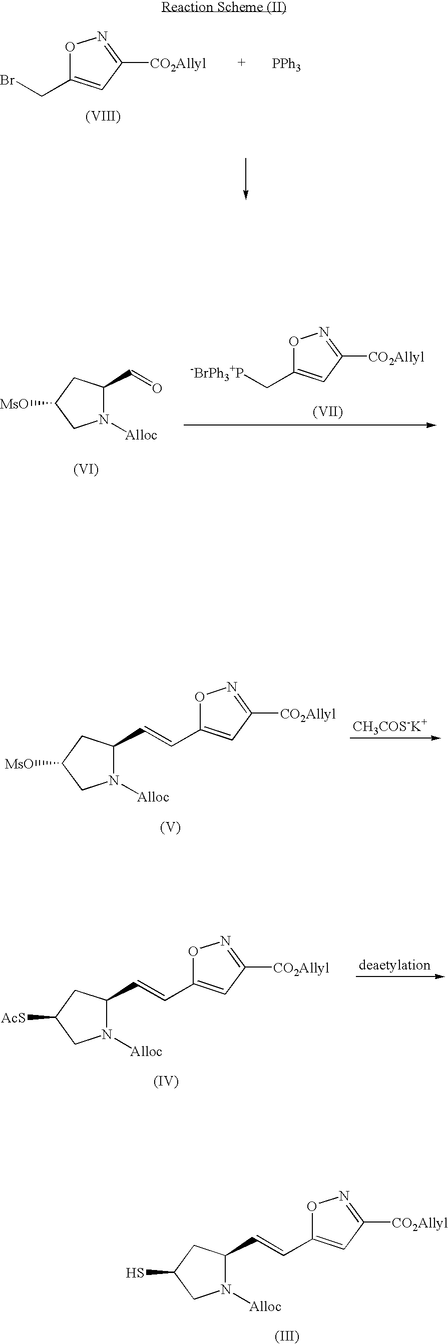
wherein,

Allyl and Alloc are the same as previously defined, Ms is methanesulfonyl, and Ac is

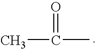

The above process comprises the steps of:

(a) subjecting the compound of formula (VIII) and triphenylphosphine to a condensation reaction to obtain the compound of formula (VII);

(b) subjecting the compounds of formula (VI) and formula (VII) to a Wittig reaction in the presence of a base to obtain the compound of formula (V);

(c) subjecting the compound of formula (V) and potassium thioacetate to a substitution reaction in a solvent to obtain the compound of formula (IV); and

(d) subjecting the compound of formula (IV) to deacetylation in a solvent to obtain the compound of formula (III).

The aldehyde of formula (VI) used as a starting material in step (b) can be prepared by the conventional method (Ohtake, N. et al. J. Antibiotics 1997, 50, 567).

Specifically, in step (a), the bromoisoxazole compound of formula (VIII) is subjected to a condensation reaction with triphenylphosphine in a solvent to obtain the triphenylphosphonium compound of formula (VII) in accordance with the conventional method (DeShong, P. et al. J. Org. Chem. 1988, 53, 1356). The solvent can be acetonitrile or dichloromethane, preferably, acetonitrile, and the reaction is carried out at the temperature ranging from 40 to 80° C., preferably, at 80° C. for 2 to 5 hours, preferably, 3 hours.

In step (b), the compound of formula (VII) is reacted in the presence of a base to obtain a ylide, and the compound of formula (VI) is reacted therewith to obtain the vinyl compound of formula (V). The base may be sodium bistrimethylsilylamine or lithium bistrimethylsilylamine, preferably, sodium bistrimethylsilylamine, and the reaction is carried out at −78° C. for 2 to 5 hours, preferably, 3 hours. The solvent used in this step is preferably tetrahydrofuran.

In step (c), the compound of formula (V) is refluxed with potassium thioacetate in a solvent to obtain the thioacetyl compound of formula (IV), for 4 to 7 hours, preferably, 5 hours, and the solvent may be a mixture of acetone and dimethylformamide, acetonitrile, acetone or dimethylformamide, preferably, a mixture of acetone and dimethylformamide (3:1 (v/v)).

In step (d), the compound of formula (IV) is deacetylated using sodium thiomethoxide in a solvent to obtain the compound of formula (III), at a temperature ranging from −10° C. to room temperature, preferably, at 0° C. for 20 to 60 minutes, preferably, 30 minutes. The solvent may be allyl alcohol.

The 1β-methylcarbapenem derivative of the present invention shows a markedly better combination of antibacterial activities against Gram-positive and Gram-negative bacteria including clinically isolated strains than known antibiotics such as imipenem, meropenem and ertapenem. It is also highly stable to DHP-I, and exhibits an in vivo half-life and bioavailability which are superior to those of the conventional drugs.

The present invention also includes within its scope a pharmaceutical composition for an antibacterial agent comprising a therapeutically effective amount of 1β-methylcarbapenem derivative of formula (I), or a pharmaceutically acceptable salt thereof as an active ingredient together with a pharmaceutically acceptable carrier.

The pharmaceutical compositions of the invention may be administered parenterally in the route of intravenous, intraperitoneal, subcutaneous and so forth, and formulated for parenteral administration such as injection in accordance with conventional methods.

The compound of formula (I) or a pharmaceutically acceptable salt thereof may be administered as an active ingredient in an effective amount ranging from about 0.1 to 100 mg/kg body weight in case of mammals including human, preferably from about 0.1 to 10 mg/kg per day in a single dose or in divided doses. However, the foregoing dosage should be monitored, and change in consideration of idiosyncrasy and weight of the patient, kind and seriousness of illnesses, characteristics of the drug and interval and duration of drug.

The following Example is intended to further illustrate the present invention without limiting its scope.

### EXAMPLE

### Preparation of (1R,5S,6S,8R,3′S,5′S)-2-{5′-[(E)-2-(3-carboxylic acid or sodium carboxylate-5-isoxazolo)ethenyl]pyrrolidin-3′-ylthio}-6-(1-hydroxyethyl)-1-methylcarbapen-2-em-3-carboxylic acid

### (Step 1) Preparation of 3-allyloxycarbonyl-5-bromomethylisoxazol (formula (VIII))

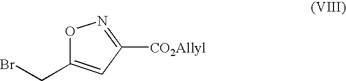

2.30 g (12.6 mmol) of 3-allyloxycarbonyl-5-hydroxymethylisoxazol was dissolved in 30 ml of anhydrous dichloromethane, cooled to −20° C., and 3.8 g (14.5 mmol) of triphenylphosphine was added thereto. 4.7 g (14.2 mmol) of carbon tetrabromide was added to the mixture at the same temperature, and stirred for 30 minutes. The resulting mixture was concentrated under a reduced pressure to remove the solvent, and the residue was subjected to column chromatography to obtain the title compound (1.75 g, 56%).

¹H NMR (300 MHz, CDCl₃) δ4.58 (m, 2H), 4.81 (m, 2H), 5.21 (m, 2H), 6.01 (m, 1H), 6.65 (s, 1H).

### (Step 2) Preparation of 3-allyloxycarbonyl-5-isoxazolomethyltriphenylphosphonium bromide (formula (VII))

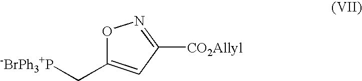

1.72 g (7 mmol) of 3-allyloxycarbonyl-5-bromomethylisoxazole prepared in step (1) was dissolved in 20 ml of acetonitrile, and 2 g (7.6 mmol) of triphenylphosphine was added thereto. The solution was refluxed for 3 hours, cooled, and the solid formed was filtered to obtain the title compound (3.2 g, 90%).

¹H NMR (300 MHz, CDCl₃) δ4.58 (m, 2H), 4.81 (m, 2H), 5.21 (m, 2H), 6.27 (d, 2H, J=14.7 Hz), 7.12 (s, 1H), 7.67 (m, 6H), 7.82 (m, 9H).

### (Step 3) Preparation of (3R,5S)-5-[(E)-2-(3-allyloxycarbonyl-5-isoxazolo)ethenyl]-3-methanesulfonyloxy-1-allyloxycarbonylpyrrolidine (formula (V))

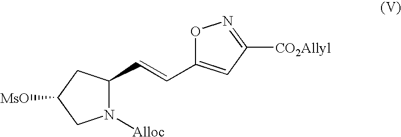

3.0 g (5.9 mmol) of 3-allyloxycarbonyl-5-isoxazolomethyltriphenylphosphonium bromide prepared in step (2) was added to 30 ml of tetrahydrofuran, and the solution was cooled to −78 ° C. 6.2 ml (6.2 mmol) of 1M sodium bistrimethylsilylamine/tetrahydrofuran was added dropwise thereto while maintaining the temperature at −78° C. and further stirred for 30 minutes at −30° C. The mixture was cooled back to −78° C., and 1.6 g (5.9 mmol) of methanesulfonyloxyformylpyrrolidine dissolved in 30 ml of tetrahydrofuran was added dropwise thereto. The reaction mixture was allowed to warm up to room temperature, stirred for 3 hours, cooled to 0° C., and saturated ammonium chloride solution was added dropwise thereto. The resulting mixture was concentrated under a reduced pressure to remove the solvent, and treated with 50 ml of water and 50 ml of dichloromethane. The dichloromethane layer was separated, dried over anhydrous magnesium sulfate, filtered, concentrated under a reduced pressure to remove the solvent, and the residue was subjected to column chromatography to obtain the title compound (1.9 g, 76%) as a colorless oil.

¹H NMR (300 MHz, CDCl₃) δ2.16 (m, 1H), 2.61 (m, 1H), 3.08 (s, 3H), 3.68-3.78 (m, 1H), 4.00 (m, 1H), 4.60 (m, 3H), 4.69 (m, 1H), 5.25-5.45 (m, 5H), 5.98 (m, 2H), 6.48-6.50 (s, 2H), 6.57 (m, 1H).

### (Step 4) Preparation of (3R,5S)-3-thioacetyl-5-[(E)-2-(3-allyloxycarbonyl-5-isoxazolo)ethenyl]-1-allyloxycarbonylpyrrolidine (formula (IV))

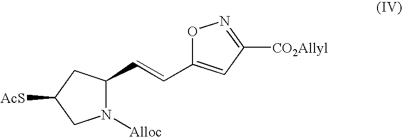

1.05 g (2.46 mmol) of (3R,5S)-5-[(E)-2-(3-allyloxycarbonyl-5-isoxazolo)ethenyl]-3-methanesulfonyloxy-1-allyloxycarbonylpyrrolidine prepared in step (3) was dissolved in 30 ml of a mixture of acetone and dimethylformamide (3:1 (v/v)), and 0.64 g (5.9 mmol) of potassium thioacetate was added thereto. The resulting mixture was refluxed for 5 hours, cooled to room temperature, and concentrated under a reduced pressure to remove the solvent. The residue was treated with 50 ml of water and 50 ml of dichloromethane. The dichloromethane layer was separated, dried over anhydrous magnesium sulfate, filtered, concentrated under a reduced pressure to remove the solvent, and the residue was subjected to column chromatography to obtain the title compound (0.75 g, 75%) as a pale yellow oil.

¹H NMR (300 MHz, CDCl₃) δ1.89 (m, 1H), 2.35 (s, 3H), 2.70 (m, 1H), 3.38 (m, 1H), 4.00-4.09 (m, 2H), 4.61 (m, 3H), 4.88 (m, 3H), 5.32-5.47 (m, 4H), 6.05 (m, 2H), 6.54 (s, 2H), 6.60 (m, 1H).

### (Step 5) Preparation of allyl (1R,5S,6S,8R,3′S,5′S)-2-{5′-[(E)-2-(3-allyloxycarbonyl-5-isoxazolo)ethenyl]-1-allyloxycarbonylpyrrolidin-3′-ylthio}-6-(1-hydroxyethyl)-1-methylcarbapen-2-em-3-carboxylate (formula (IX))

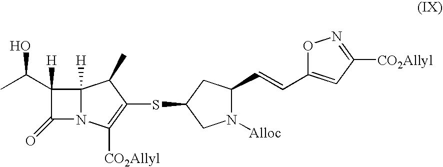

0.55 g (1.36 mmol) of (3R,5S)-3-thioacetyl-5-[(E)-2-(3-allyloxycarbonyl-5-isoxazolo)ethenyl]-1-allyloxycarbonylpyrrolidine prepared in step (4) was dissolved in 10 ml of allyl alcohol, cooled to 0° C., and 0.10 g (1.50 mmol) of sodium thiomethoxide was added dropwise thereto. The resulting mixture was stirred for 30 minutes at the same temperature, and 1.5 ml of 1N hydrochloric acid was added thereto, to make the acidic solution. The resulting solution was concentrated under a reduced pressure to remove the solvent, and extracted with 50 ml of ethyl acetate. The extract was washed with saturated sodium carbonate, and the aqueous layer was extracted with 50 ml of ethyl acetate. The combined organic layer was dried over anhydrous magnesium sulfate, filtered, concentrated under a reduced pressure to obtain the compound of formula (III), which was used in the following step without further purification.

0.67 g (1.36 mmol) of allyl (1R,5S,6S,8R)-2-diphenylphosphoryloxy-6-(1-hydroxyethyl)-1-methylcarbapen-2-em-3-carboxylate of formula (II) was dissolved in 50 ml of acetonitrile under a nitrogen atmosphere. 0.28 ml (1.64 mmol) of N,N-diisopropylethylamine was added thereto at 0° C., and to the resulting mixture 0.46 g (1.36 mmol) of the compound of formula (III) obtained above dissolved in 10 ml of acetonitrile was added. The mixture was stirred at the same temperature for 1.5 hours, and treated with 50 ml of ethyl acetate and 100 ml of saturated sodium chloride solution. The organic layer was separated, dried over anhydrous magnesium sulfate, filtered, concentrated under a reduce pressure, and the resulting residue was subjected to column chromatography to obtain the title compound (0.48 g, 65%) as a pale yellow foam.

¹H NMR (300 MHz, CDCl₃) δ1.28 (d, 3H, J=7.2 Hz), 1.36 (d, 3H, J=6.2 Hz), 1.89 (m, 1H), 2.18 (m, 1H), 2.74 (m, 1H), 3.28 (m, 1H), 3.40 (m, 2H), 3.73 (m, 1H), 4.18 (m, 1H), 4.25 (m, 2H), 4.58-4.89 (m, 7H), 5.24-5.48 (m, 6H), 5.96 (m, 3H), 6.56 (m, 3H).

### (Step 6) Preparation of (1R,5S,6S,8R,3′S,5′S)-2-{5′-[(E)-2-(3-carboxylic acid or sodium carboxylate-5-isoxazolo)ethenyl]pyrrolidin-3 ′-ylthio}-6-(1-hydroxyethyl)-1-methylcarbapen-2-em-3 -carboxylic acid (formula (I))

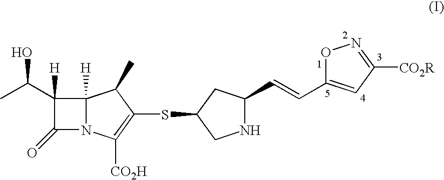

100 mg (0.17 mmol) of allyl (1R,5S,6S,8R,3′S,5′S)-2-{5′-[(E)-2-(3-allyloxycarbonyl-5-isoxazolo)ethenyl]-1-allyloxycarbonylpyrrolidin-3′-ylthio}-6-(1-hydroxyethyl)-1-methylcarbapen-2-em-3-carboxylate prepared in step (5) was dissolved in 2 ml of dichloromethane under a nitrogen atmosphere. 6.0 mg (0.0052 mmol) of tetrakis(triphenylphosphine)palladium [0] was added thereto at 0° C., and 0.093 ml (0.35 mmol) of tributyltin hydride was added dropwise thereto. The solution was stirred at the same temperature for 1.5 hours to obtain the (E)-2-(3-carboxylic acid-5-isoxazolo)ethenyl compound.

To obtain the (E)-2-(3-sodium carboxylate-5-isoxazolo)ethenyl compound, 0.042 g (0.26 mmol) of sodium 2-ethylhexanoate was added to the above reaction solution, and stirred for 30 minutes. The mixture was washed with water, and extracted with ethyl acetate. The aqueous layer was freeze-dried, and the residue was purified by Diaion HP-20 column chromatography (3% aqueous tetrahydrofuran solution) to obtain the title compound (41.7 mg, 52%) as a white solid.

mp: 243-245° C.

IR (KBr): 3390, 2968, 1748, 1614 cm⁻¹

¹H NMR (300 MHz, D₂O) δ1.09 (d, 3H, J=7.1 Hz), 1.15 (d, 3H, J=6.3 Hz), 1.59 (m, 1H), 2.59 (m, 1H), 3.07 (m, 1H), 3.18-3.32 (m, 1H), 3.39 (m, 1H), 3.82 (m, 1H), 3.99 (m, 1H), 4.06-4.13 (m, 2H), 6.46-6.62 (m, 3H).

¹³C NMR (75 MHz, D₂O) δ176.4, 168.2, 167.8, 166.2, 161.4, 140.0, 132.5, 128.7, 118.8, 102.3, 65.1, 60.5, 58.5, 55.9, 53.0, 42.7, 40.5, 36.4, 20.0, 15.0.

FABHRMS (m/z) Calcd for C₂₀H₂₂N₃O₇SNa₂ (M+Na)⁺: 494.0975, Found: 494.0974.

### TEST EXAMPLE 1

### Antibacterial Activity Test

The in vitro antibacterial activities of the sodium 3-carboxylate compound of the present invention prepared in the above Example were measured against standard strains (Table 1), clinically isolated aerobic Gram-positive strains (Table 2), clinically isolated aerobic Gram-negative strains (Table 3), clinically isolated anaerobic Gram-positive strains (Table 4) and clinically isolated anaerobic Gram-negative strains (Table 5) using Gram-positive bacteria such as Streptococcus and Staphylococcus, Gram-negative bacteria such as Escherichia, Salmonella, Krebsiella and Enterobacter. Imipenem (IPM), meropenem (MPM) and ertapenem (EPM) were used as control groups.

Specifically, the test compound was serially double diluted and added to each bacteria strain cultured in a diluted agar culture medium, and incubated at 37° C. for 18 to 20 hours to determine the minimum inhibitory concentration (MIC) at which the growth of each strain was inhibited. The results are shown in Tables 1 to 5, and MIC₅₀ and MIC₉₀ represent the concentrations at which the test strain's growth was inhibited to the extents of 50% and 90%, respectively.

TABLE 1
MIC against standard strains

| | Minimum Inhibitory Concentration | | |
|---|---|---|---|
| | (MIC, μg/ml) | | |
| strain | Example | IPM | MPM |
| Streptococcus pyogenes 308A | 0.049 | 0.004 | 0.007 |
| Streptococcus pyogenes 77A | 0.049 | <0.002 | 0.007 |
| Streptococcus faecium MD 8b | 12.50 | 0.781 | 12.50 |
| Staphylococcus aureus SG511 | 0.098 | 0.013 | 0.098 |
| Staphylococcus aureus 285 | 0.195 | 0.013 | 0.195 |
| Staphylococcus aureus 503 | 0.098 | 0.007 | 0.098 |
| Escherichia coli 078 | 0.025 | 0.098 | 0.025 |
| Escherichia coli DC 0 | 0.025 | 0.195 | 0.025 |
| Escherichia coli DC 2 | 0.025 | 0.195 | 0.025 |
| Escherichia coli TEM | 0.025 | 0.098 | 0.025 |
| Escherichia coli 1507E | 0.025 | 0.098 | 0.025 |
| Pseudomonas aeruginosa 9027 | 0.098 | 0.391 | 0.195 |
| Pseudomonas aeruginosa 1592E | 0.195 | 0.781 | 0.098 |
| Pseudomonas aeruginosa 1771 | 0.391 | 0.781 | 0.391 |
| Pseudomonas aeruginosa 1771M | 0.391 | 0.195 | 0.098 |
| Salmonella typhimurium | 0.049 | 0.781 | 0.049 |
| Klebsiella cxytoca 1082E | 0.049 | 0.195 | 0.049 |
| Klebsiella aerogenes 1522E | 0.049 | 0.195 | 0.049 |
| Enterobacter cloacae P99 | 0.098 | 0.098 | 0.049 |
| Enterobacter cloacae 1321E | 0.025 | 0.098 | 0.025 |

TABLE 2
MIC against clinically isolated aerobic Gram-positive strains

| Organism | Anti- | MIC (μg/ml) | | |
|---|---|---|---|---|
| (No. of strain) | biotics | MIC Range | MIC₅₀ | MIC₉₀ |
| Methicillin-sensitive | Example | 0.06-0.12 | 0.12 | 0.12 |
| Staphylococcus aureus | IPM | 0.015-0.06 | 0.015 | 0.03 |
| (33) | MPM | 0.06-0.25 | 0.12 | 0.12 |
| | EPM | 0.25-0.5 | 0.25 | 0.25 |
| Staphylococcus coagulase | Example | 0.06-0.5 | 0.12 | 0.25 |
| (22) | IPM | 0.008-0.03 | 0.015 | 0.015 |
| | MPM | 0.03-0.5 | 0.06 | 0.12 |
| | EPM | 0.12-1 | 0.25 | 0.5 |
| Streptococcus pyogenes | Example | <0.008 | <0.008 | <0.008 |
| (15) | IPM | <0.008 | <0.008 | <0.008 |
| | MPM | <0.008 | <0.008 | <0.008 |
| | EPM | 0.008-0.015 | 0.15 | 0.15 |
| Streptococcus agalactiae | Example | 0.008-0.015 | 0.008 | 0.015 |
| (15) | IPM | 0.008-0.015 | 0.008 | 0.015 |
| | MPM | 0.03 | 0.03 | 0.03 |
| | EPM | 0.03-0.06 | 0.06 | 0.06 |
| Streptococcus pneumoniae | Example | 0.008-0.25 | 0.008 | 0.12 |
| (22) | IPM | 0.008-0.5 | 0.12 | 0.25 |
| | MPM | 0.008-0.5 | 0.5 | 0.5 |
| | EPM | 0.008-1 | 0.5 | 1 |
| Enterococcus faecalis | Example | 4-32 | 8 | 32 |
| (30) | IPM | 0.5-4 | 1 | 4 |
| | MPM | 2-16 | 4 | 16 |
| | EPM | 4-64 | 16 | 32 |
| Enterococcus faecium | Example | 16-128 | 128 | 128 |
| (29) | IPM | 2-128 | 128 | 128 |
| | MPM | 16-128 | 128 | 128 |
| | EPM | 32-128 | 128 | 128 |

TABLE 3
MIC against clinically isolated aerobic Gram-negative strains

| Organism | Anti- | MIC (μg/ml) | | |
|---|---|---|---|---|
| (No. of strain) | biotics | MIC Range | MIC₅₀ | MIC₉₀ |
| Moraxella | Example | 0.015-0.06 | 0.03 | 0.06 |
| catarrhalis | IPM | 0.008-0.25 | 0.06 | 0.06 |
| (24) | MPM | 0.008-0.03 | 0.008 | 0.008 |
| | EPM | 0.008-0.12 | 0.015 | 0.03 |
| Haemophilus | Example | 0.25-8 | 0.25 | 4 |
| influenzae | IPM | 0.25-8 | 1 | 4 |
| (24) | MPM | 0.06-1 | 0.25 | 1 |
| | EPM | 0.12-1 | 0.12 | 0.5 |
| Escherichia | Example | 0.008-2 | 0.03 | 0.25 |
| coli | IPM | 0.06-1 | 0.12 | 0.5 |
| (30) | MPM | 0.008-0.5 | 0.015 | 0.03 |
| | EPM | 0.008-4 | 0.008 | 0.12 |
| Citrobacter | Example | 0.015-0.25 | 0.03 | 0.12 |
| freundii | IPM | 0.06-0.5 | 0.12 | 0.5 |
| (14) | MPM | 0.015-0.06 | 0.015 | 0.03 |
| | EPM | 0.008-0.5 | 0.008 | 0.25 |
| Klebsiella | Example | 0.015-0.25 | 0.03 | 0.12 |
| pneumoniae | IPM | 0.06-1 | 0.12 | 0.5 |
| (30) | MPM | 0.015-0.06 | 0.03 | 0.06 |
| | EPM | 0.008-1 | 0.03 | 0.5 |
| Klebsiella | Example | 0.015-0.25 | 0.03 | 0.03 |
| oxytoca | IPM | 0.06-0.5 | 0.12 | 0.5 |
| (15) | MPM | 0.015-0.06 | 0.03 | 0.03 |
| | EPM | 0.008-0.25 | 0.008 | 0.008 |
| Enterobacter | Example | 0.015-2 | 0.12 | 0.5 |
| cloacae | IPM | 0.12-1 | 0.25 | 1 |
| (29) | MPM | 0.015-0.5 | 0.03 | 0.25 |
| | EPM | 0.015-2 | 0.12 | 2 |
| Enterobacter | Example | 0.015-0.12 | 0.06 | 0.25 |
| aerogenes | IPM | 0.12-0.5 | 0.12 | 0.5 |
| (14) | MPM | 0.015-0.06 | 0.03 | 0.06 |
| | EPM | 0.008-0.5 | 0.06 | 0.5 |
| Serratia | Example | 0.03-16 | 0.03 | 16 |
| marcescens | IPM | 0.12-4 | 0.25 | 2 |
| (14) | MPM | 0.03-8 | 0.03 | 8 |
| | EPM | 0.015-16 | 0.06 | 16 |
| Proteus | Example | 0.015-0.06 | 0.03 | 0.06 |
| mirabilis | IPM | 0.25-4 | 2 | 2 |
| (15) | MPM | 0.015-0.06 | 0.06 | 0.06 |
| | EPM | 0.008-0.015 | 0.008 | 0.015 |
| Proteus | Example | 0.03-0.06 | 0.06 | 0.06 |
| vulgaris | IPM | 0.25-2 | 1 | 2 |
| (15) | MPM | 0.03-0.06 | 0.06 | 0.06 |
| | EPM | 0.008-0.03 | 0.015 | 0.015 |
| Morganella | Example | 0.03-0.12 | 0.03 | 0.06 |
| morganii | IPM | 0.5-2 | 1 | 2 |
| (15) | MPM | 0.03-0.12 | 0.06 | 0.12 |
| | EPM | 0.008-0.03 | 0.008 | 0.03 |
| Providentia | Example | 0.008-8 | 0.06 | 8 |
| sp. | IPM | 0.25-4 | 2 | 2 |
| (13) | MPM | 0.015-2 | 0.06 | 2 |
| | EPM | 0.008-16 | 0.03 | 16 |
| Acinetobacter | Example | 2-128 | 8 | 64 |
| baumannii | IPM | 0.25-32 | 1 | 8 |
| (30) | MPM | 0.25-64 | 1 | 8 |
| | EPM | 4-128 | 8 | 64 |
| Pseudomonas | Example | 0.06-128 | 4 | 64 |
| aeruginosa | IPM | 0.5-128 | 2 | 16 |
| (60) | MPM | 0.06-128 | 2 | 16 |
| | EPM | 1-128 | 32 | 128 |

TABLE 4
MIC against clinically isolated anaerobic Gram-positive strains

| Organism | Anti- | MIC (μg/ml) | | |
|---|---|---|---|---|
| (No. of strain) | biotics | MIC Range | MIC₅₀ | MIC₉₀ |
| Peptostreptococcus spp. | Example | 0.06-4 | 0.12 | 4 |
| (27) | IPM | 0.06-2 | 0.06 | 2 |
| | MPM | 0.06-4 | 0.06 | 4 |
| | EPM | 0.06-4 | 0.12 | 4 |
| Clostridium perfringens | Example | 0.06-0.12 | 0.06 | 0.12 |
| (13) | IPM | 0.06-0.12 | 0.06 | 0.12 |
| | MPM | <0.06 | <0.06 | <0.06 |
| | EPM | 0.06-0.12 | 0.06 | 0.12 |
| Clostridium difficile | Example | 2-4 | 4 | 4 |
| (15) | IPM | 4-16 | 8 | 8 |
| | MPM | 1-2 | 1 | 2 |
| | EPM | 4-8 | 4 | 8 |

TABLE 5
MIC against clinically isolated anaerobic Gram-negative strains

| Organism | | MIC (μg/ml) | | |
|---|---|---|---|---|
| (No. of strain) | Antibiotics | MIC Range | MIC₅₀ | MIC₉₀ |
| Bacteroides fragilis | Example | 0.25-4 | 0.5 | 1 |
| (34) | IPM | 0.06-2 | 0.25 | 0.5 |
| | MPM | 0.12-4 | 0.12 | 0.25 |
| | EPM | 0.12-4 | 0.25 | 1 |
| Bacteroides | Example | 0.5-8 | 1 | 4 |
| thetaiotaomicron | IPM | 0.12-16 | 0.5 | 4 |
| (15) | MPM | 0.25-2 | 0.25 | 0.5 |
| | EPM | 0.25-8 | 2 | 2 |
| Bacteroides spp. | Example | 1-2 | 1 | 1 |
| (11) | IPM | 0.25-2 | 0.5 | 1 |
| | MPM | 0.12-0.5 | 0.5 | 0.5 |
| | EPM | 0.5-2 | 1 | 2 |

As can be seen in Table 1, the sodium 3-carboxylate compound prepared in Example showed excellent antibacterial activity against Gram-positive and Gram-negative strains, like meropenem.

The results in Table 2 show that the sodium 3-carboxylate compound of Example exhibited excellent antibacterial activities against all strains except Enterococcus faecium, and it was a better against Streptococcus pneumoniae than the control compounds. It also showed inhibitory activities against aerobic Gram-negative strains which were equivalent to those of IPM and MPM as shown in Table 3, and the inventive compound effectively inhibited the growth of anaerobic Gram-positive and Gram-negative strains as shown in Tables 4 and 5.

Thus, the compound of the present invention has a far more desirable combination of antibacterial activities against clinically isolated Gram-positive and Gram-negative strains than any of the existing carbapenem antibiotics.

### TEST EXAMPLE 2

### Stability to DHP-I

To investigate the stability of sodium 3-carboxylate compound of formula (I) prepared in Example to DHP-I secreted in the kidney, the following experiment was performed.

DHP-I used in the experiment was isolated from the kidney cortex of a porcine. The enzyme quantity at which the concentration of imipenem was reduced by one half by hydrolysis at 30° C. for 30 minutes was defined as one unit. 50 μg/ml of a test drug and a unit of DHP-I were added to 1 ml of MOPS buffer (pH 7.0), the mixture was maintained at 30° C. and OD values at 299 nm were measured after 0.5, 1, 2, 4 hours.

The half-life of meropenem in the presence of DHP-I was defined as 1.00, and the relative stability of each drug was measured using imipenem (IPM) and meropenem (MPM) as controls. The results are shown in Table 6.

TABLE 6

| Drug | Example | IPM | MPM |
|---|---|---|---|
| DHP-I stability | 4.57 | 0.18 | 1.00 |

As can be seen in Table 6, the sodium 3-carboxylate compound of Example showed an about 25-fold higher stability than imipenem, and an about 4.5-fold higher stability than meropenem. Thus, the compound of Example is much more bioavailable than the controls.

### TEST EXAMPLE 3

### Pharmacokinetics Test

The pharmacokinetic behavior of the sodium 3-carboxylate compound of Example was determined as follows.

Male Sprague-Dawley rats (weighing 250 g, 14-15 weeks old, 5 rats/group) and Beagle dogs (weighing 10 kg, 3 dogs/group) were maintained by feeding conventional hard food at identical conditions for more than 7 days. The test animals were fasted except water for more than 24 hours before tested.

Each of the compounds of Example and meropenem was dissolved in distilled water, and intravenously injected at a dosage of 20 mg/kg body weight to rats and 5 mg/kg body weight to dogs, respectively. Blood samples were withdrawn from the animals at 0.25, 0.5, 0.75, 1, 2, 3, 4, 8, 12 and 24 hours after the injection.

500 μl of each blood sample was centrifuged at 12,000 rpm for 30 seconds, the supernatant was filtered through a 0.22 μm filter, and analyzed by HPLC/UV, and the result was listed in Table 7.

Column: Symmetry (5 μm, 23.9×150 mm, Waters, USA)

Mobile phase: 30 mM phosphate buffer (pH 3.0): acetonitrile=85:15

Volume: 30 μl

Flow rate: 0.5 ml/min

Detection: UV 260 nm (for Example) and 298 nm (for MPM)

TABLE 7

| | rat | | dog | |
|---|---|---|---|---|
| | Example | MPM | Example | MPM |
| | (20 mg/kg) | (20 mg/kg) | (5 mg/kg) | (5 mg/kg) |
| T_{1/2}(min) | 12.4 ± 4.1 | 4.0 ± 0.2 | 41 | 33 |
| AUC | 1519 ± 168 | 383 ± 36 | 861 | 695 |
| (μg · min/ml) | | | | |
| CL | 13.3 ± 1.5 | 54.2 ± 5.3 | — | — |
| (ml/min/kg) | | | | |

As can be seen in Table 7, the sodium 3-carboxylate compound of Example showed about an about 3-fold longer half-life and an about 4-fold higher bioavailability than meropenem in rats. Its half-life and bioavailability observed for dogs were also excellent.

While the invention has been described with respect to the above specific embodiments, it should be recognized that various modifications and changes may be made and also fall within the scope of the invention as defined by the claims that follow.

What is claimed is:

1. The 1β-methylcarbapenem derivative of formula (I) or a pharmaceutically acceptable salt thereof.

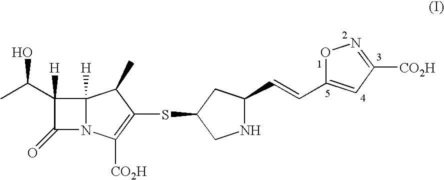

2. The derivative of claim 1, wherein the salt is a sodium salt.

3. A process for the preparation of the derivative of claim 1, which comprises the steps of:
(a) reacting the compounds of formula (II) and formula (III) in the presence of a base to obtain the protected carbapenem compound of formula (IX); and
(b) subjecting the compound of formula (IX) to a deprotection reaction. 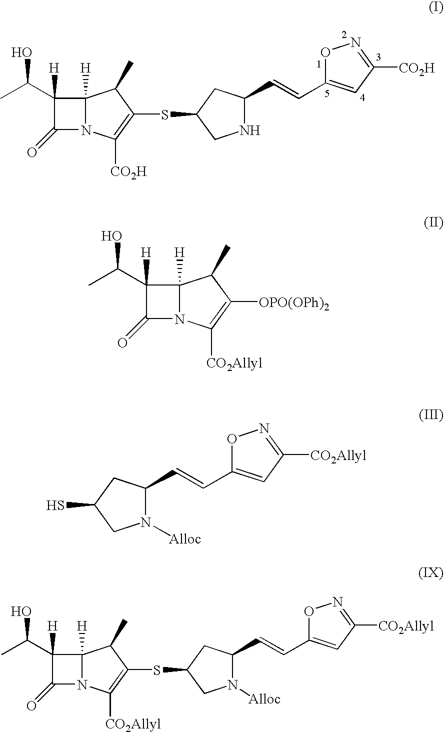
wherein,
Allyl is —CH₂—CH═CH₂ and Alloc is

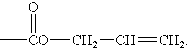

4. The process of claim 3, wherein the base used in step (a) is selected from the group consisting of trimethylamine, triethylamine, N,N-diisopropylethylamine, 2,6-lutidine, picoline, N,N-dimethylaniline, pyridine and 4-dimethylaminopyridine.

5. The process of claim 3, wherein step (a) is conducted in acetonitrile.

6. The process of claim 3, wherein step (a) is carried out at a temperature ranging from −10 to 10° C. for 1 to 3 hours.

7. The process of claim 3, wherein the deprotection is carried out by reacting the compounds of formula (IX) with tributyltin hydride in the presence of a catalyst selected from the group consisting of tetrakis(triphenylphosphine)palladium and di(triphenylphosphine)dichloropalladium.

8. The process of claim 3, wherein the deprotection is carried out at a temperature ranging from −10 to 10° C. for 1 to 3 hours in a solvent selected from the group consisting of dichloromethane, a mixture of carbon dichloride dichloromethane and water, and tetrahydrofuran.

9. A process for preparing the sodium salt of claim 2, which comprises reacting the compound of formula (I) with sodium 2-ethylhexanoate (SHE) or sodium bicarbonate.

10. The process of claim 9, wherein the preparation of the sodium salt is carried out at a temperature ranging from −10 to 10° C. for 10 to 60 minutes.

11. The thiol derivative of formula (III), which is used for the preparation of the compound of claim 1. 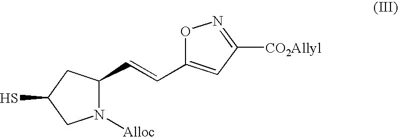
wherein,
Allyl is —CH₂—CH═CH₂ and Alloc is

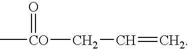

12. A process for the preparation of the thiol derivative of formula (III) of claim 11, which comprises the steps of:
(a) subjecting the compound of formula (VIII) and triphenylphosphine to a condensation reaction to obtain the compound of formula (VII);
(b) subjecting the compounds of formula (VI) and formula (VII) to a Wittig reaction in the presence of a base and a solvent to obtain the compound of formula (V);
(c) subjecting the compound of formula (V) and potassium thioacetate to a substitution reaction in a solvent to obtain the compound of formula (IV); and
(d) subjecting the compound of formula (IV) to deacetylation in a solvent to obtain the compound of formula (III). 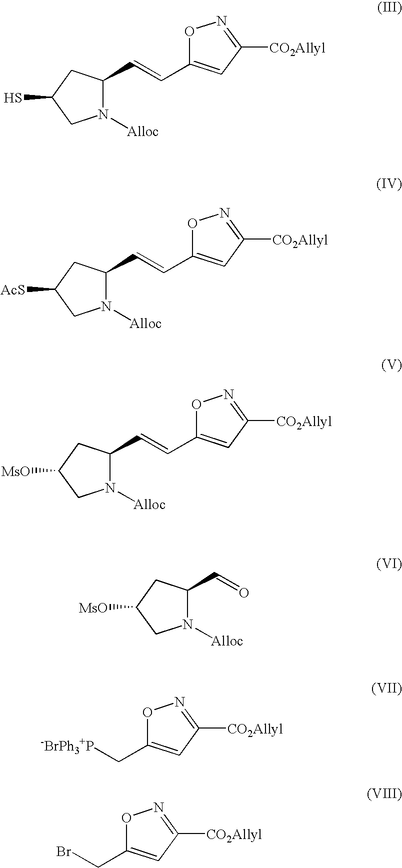
wherein,
Allyl is —CH₂—CH═CH₂, Alloc is 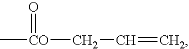
Ms is methanesulfonyl, and Ac is

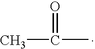

13. The process of claim 12, wherein the condensation reaction is carried out in acetonitrile or dichloromethane at a temperature ranging from 40 to 80° C. for 2 to 5 hours.

14. The process of claim 12, wherein the base used in step (b) is sodium bistrimethylsilylamine or lithium bistrimethylsilylamine.

15. The process of claim 12, wherein the solvent used in step (b) is tetrahydrofuran.

16. The process of claim 12, wherein the Wittig reaction is carried out at a temperature ranging from −78° C. for 2 to 5 hours.

17. The process of claim 12, wherein the solvent used in step (c) is acetonitrile, acetone, dimethylformamide, or a mixture thereof.

18. The process of claim 12, wherein the substitution reaction is carried out by refluxing for 4 to 7 hours.

19. The process of claim 12, wherein the solvent used in step (d) is allyl alcohol.

20. The process of claim 12, wherein the deacetylation reaction is carried out using sodium thiomethoxide.

21. The process of claim 12, the deacetylation is carried out at a temperature ranging from −10° C. to room temperature for 20 to 60 minutes

22. A pharmaceutical composition comprising the 1β-methylcarbapenem derivative of the claim 1 or a pharmaceutically acceptable salt thereof as an active antibacterial ingredient.
